(19) United States
(12) Patent Application Publication
(10) Pub. No.: US 2020/0158706 A1
(43) Pub. Date: May 21, 2020

(54) METHODS FOR ANALYSING ELECTRONIC CIGARETTE SMOKE

(71) Applicant: Micromass UK Limited, Wilmslow (GB)

(72) Inventors: Steven Derek Pringle, Hoddlesden, Darwen (GB); Emrys Jones, Manchester (GB); Michael Raymond Morris, Hadfield, Glossop (GB); Zoltán Takáts, Haslingfield, Cambridge (GB)

(21) Appl. No.: 16/072,623

(22) PCT Filed: Jan. 19, 2017
(86) PCT No.: PCT/GB2017/050131

§ 371 Date: Jul. 25, 2018

(30) Foreign Application Priority Data
Jan. 25, 2016 (GB) 1601319.5

(51) Int. Cl. G01N 33/00

(52) U.S. Cl. CPC G01N 33/0047

(57) ABSTRACT

A method of analysing a sample is disclosed that comprises activating an electronic cigarette (“e-cigarette”) or other vaporising or atomising device such that the electronic cigarette or other device emits aerosol, smoke, vapour and/or droplets, mass analysing the aerosol, smoke, vapour and/or droplets, and characterising the aerosol, smoke, vapour and/or droplets based on the mass analysis.

### CROSS-REFERENCE TO RELATED APPLICATION

This application claims priority from and the benefit of United Kingdom patent application No. 1601319.5 filed on 25 Jan. 2016. The entire content of this application is incorporated herein by reference.

### FIELD OF THE INVENTION

The present invention relates generally to mass spectrometers and in particular to the analysis of aerosol, smoke, vapour and/or droplets by mass spectrometry.

### BACKGROUND

It is desirable for electronic cigarettes (“e-cigarettes”) and similar vaporising or atomising products to undergo tests in view of quality, health and authenticity considerations.

Measuring the chemical composition of the vapour inhaled by a user presents a novel challenge to the analyst, and currently there is no convenient solution. Typical current methodologies require the trapping of components of the emitted vapour either in a solvent or on a filter paper, prior to an extraction step and chromatography-mass spectrometry analysis. However, such methods are laborious and expensive.

It is desired to provide an improved method of analysing a sample, and in particular an improved method of analysing a sample which may comprise aerosol, smoke, vapour and/or droplets emitted by an electronic cigarette (“e-cigarette”) or similar vaporising or atomising product.

### SUMMARY

According to an aspect there is provided a method of analysing a sample comprising:

activating an electronic cigarette (“e-cigarette”) or other vaporising or atomising device such the electronic cigarette or other device emits aerosol, smoke, vapour and/or droplets;

mass analysing the aerosol, smoke, vapour and/or droplets; and

characterising the aerosol, smoke, vapour and/or droplets based on the mass analysis.

Various embodiments described herein are directed to methods of analysing a sample in which an electronic cigarette (“e-cigarette”) or other vaporising or atomising device is activated such that the electronic cigarette or other device emits an aerosol, smoke, vapour and/or droplets, and then the aerosol, smoke, vapour and/or droplets is mass analysed.

The various embodiments described herein present a simple and fast method of analysing the chemical content of the aerosol, vapour or smoke emitted by electronic cigarettes and similar vaporising or atomising devices. In particular, the various embodiments effectively provide a single-step sampling methodology, in contrast to the typical multi-step methodologies described above. Various embodiments provide direct, substantially instantaneous profiles of electronic cigarette smoke or vapour.

Furthermore, the Applicants have found that mass analysing the aerosol, smoke, vapour and/or droplets emitted by an electronic cigarette or other vaporising or atomising device is particularly useful for the characterisation of the aerosol, smoke, vapour and/or droplets. In particular, the Applicants have found that sampling the aerosol, smoke, vapour and/or droplets from an electronic cigarette produces an information rich and characteristic mass spectrum. Contaminants can be detected and different compositions or blends of electronic cigarette solution (or “e-liquid”) can be differentiated.

It will be appreciated, therefore, that the various embodiments described herein provide an improved method of analysing a sample.

Activating the electronic cigarette or other device may comprise activating an activation switch or button of the electronic cigarette or other device.

Activating the electronic cigarette or other device may comprise causing a gas to pass through the electronic cigarette or other device.

Activating the electronic cigarette or other device may comprise causing a pulse of gas to pass through the electronic cigarette or other device.

Activating the electronic cigarette or other device may comprise activating the electronic cigarette or other device such that an electronic cigarette solution (“e-liquid”) is vaporised by the electronic cigarette or other device so as to produce the aerosol, smoke, vapour and/or droplets.

The method may comprise positioning the electronic cigarette or other device in a sampling or inlet port, wherein the sampling or inlet port is arranged and adapted to accommodate the electronic cigarette or other device.

The method may comprise positioning a mouthpiece of the electronic cigarette or other device in the sampling or inlet port, wherein the sampling or inlet port is arranged and adapted to accommodate a mouthpiece of an electronic cigarette or other device.

The method may comprise providing an airtight seal between the electronic cigarette or other device and the sampling or inlet port.

Mass analysing the aerosol, smoke, vapour and/or droplets may comprise generating analyte ions from the aerosol, smoke, vapour and/or droplets.

Generating analyte ions from the aerosol, smoke, vapour and/or droplets may comprise causing the aerosol, smoke, vapour and/or droplets to impact a collision surface.

Mass analysing the aerosol, smoke, vapour and/or droplets may comprise mass analysing the analyte ions or ions derived from the analyte ions.

Characterising the aerosol, smoke, vapour and/or droplets may comprise determining the chemical composition of the aerosol, smoke, vapour and/or droplets based on the mass analysis.

Characterising the aerosol, smoke, vapour and/or droplets may comprise identifying one or more contaminants in the aerosol, smoke, vapour and/or droplets based on the mass analysis.

Characterising the aerosol, smoke, vapour and/or droplets may comprise identifying one or more compositions of electronic cigarette solution (“e-liquid”) based on the mass analysis.

The method may comprise:

activating a plurality of electronic cigarettes (“e-cigarettes”) or other vaporising or atomising devices such that each of the plurality of electronic cigarettes or other devices emits aerosol, smoke, vapour and/or droplets;

mass analysing the aerosol, smoke, vapour and/or droplets emitted from each of the plurality of electronic cigarettes or other devices; and

characterising the plurality of electronic cigarettes or other devices based on the mass analyses.

The method may be performed automatically without user interaction.

According to another aspect there is provided apparatus for analysing a sample comprising:

one or more first devices arranged and adapted to activate an electronic cigarette (“e-cigarette”) or other vaporising or atomising device such that the electronic cigarette or other device emits aerosol, smoke, vapour and/or droplets;

a mass analyser arranged and adapted to mass analyse the aerosol, smoke, vapour and/or droplets; and

one or more second devices arranged and adapted to characterise the aerosol, smoke, vapour and/or droplets based on the mass analysis.

The one or more first devices may be arranged and adapted to activate the electronic cigarette or other device by activating an activation switch or button of the electronic cigarette or other device.

The one or more first devices may be arranged and adapted to activate the electronic cigarette or other device by causing a gas to pass through the electronic cigarette or other device.

The one or more first devices may be arranged and adapted to activate the electronic cigarette or other device by causing a pulse of gas to pass through the electronic cigarette or other device.

The one or more first devices may be arranged and adapted to activate the electronic cigarette or other device such that an electronic cigarette solution (“e-liquid”) is vaporised by the electronic cigarette or other device so as to produce the aerosol, smoke, vapour and/or droplets.

The apparatus may comprise a sampling or inlet port, wherein the sampling or inlet port is arranged and adapted to accommodate the electronic cigarette or other device.

The sampling or inlet port may be arranged and adapted to accommodate a mouthpiece of an electronic cigarette or other device.

The sampling or inlet port may be arranged and adapted to provide an airtight seal between the electronic cigarette or other device and the sampling or inlet port.

The apparatus may comprise one or more devices arranged and adapted to generate analyte ions from the aerosol, smoke, vapour and/or droplets.

The apparatus may comprise a collision surface or gas, wherein the apparatus is arranged and adapted to cause the aerosol, smoke, vapour and/or droplets to impact the collision surface or gas to generate analyte ions.

The mass analyser may be arranged and adapted to mass analyse the analyte ions or ions derived from the analyte ions.

The one or more second devices may be arranged and adapted to characterise the aerosol, smoke, vapour and/or droplets by determining the chemical composition of the aerosol, smoke, vapour and/or droplets based on the mass analysis.

The one or more second devices may be arranged and adapted to characterise the aerosol, smoke, vapour and/or droplets by identifying one or more contaminants in the aerosol, smoke, vapour and/or droplets based on the mass analysis.

The one or more second devices may be arranged and adapted to characterise the aerosol, smoke, vapour and/or droplets by identifying one or more compositions of electronic cigarette solution (“e-liquid”) based on the mass analysis.

The apparatus may comprise:

one or more devices arranged and adapted to activate a plurality of electronic cigarettes (“e-cigarettes”) or other vaporising or atomising devices such that each of the plurality of electronic cigarettes or other devices emits aerosol, smoke, vapour and/or droplets;

one or more mass analysers arranged and adapted to mass analyse the aerosol, smoke, vapour and/or droplets emitted from each of the plurality of electronic cigarettes or other devices; and

one or more devices arranged and adapted to characterise the plurality of electronic cigarettes or other devices based on the mass analyses.

The apparatus may be arranged and adapted to operate automatically without user interaction.

According to another aspect there is provided a method of analysing a sample comprising:

causing a cigarette to emit smoke;

impacting the smoke with a collision surface to produce analyte ions;

mass analysing the smoke, the analyte ions and/or ions derived from the analyte ions; and

characterising the smoke based on the mass analysis.

According to another aspect there is provided apparatus for analysing a sample comprising:

one or more devices arranged and adapted to cause a cigarette to emit smoke;

a collision surface, wherein the apparatus is arranged and adapted to impact the smoke with the collision surface to produce analyte ions;

a mass analyser arranged and adapted to mass analyse the smoke, the analyte ions and/or ions derived from the analyte ions; and

one or more devices arranged and adapted to characterise the smoke based on the mass analysis.

The apparatus may comprise a mass spectrometer.

### BRIEF DESCRIPTION OF THE DRAWINGS

Various embodiments will now be described, by way of example only, and with reference to the accompanying drawings in which:

FIG. 1 illustrates the triggering of two types of electronic cigarette and their coupling to a mass spectrometer;

FIG. 2 shows a mass spectrometer according to an embodiment;

FIG. 3 shows a mass spectrometer according to an embodiment

FIG. 4 shows mass spectra in positive and negative ion mode for a single “burn” of an electronic cigarette obtained in accordance with an embodiment;

FIG. 5 shows the results of analysis differentiating two different types of electronic cigarette solution in accordance with an embodiment;

FIG. 6 shows mass spectra in positive ion mode for a single “burn” of an electronic cigarette obtained in accordance with an embodiment; and

FIG. 7 shows plural cigarette sampling devices.

### DETAILED DESCRIPTION

The recent growth of electronic cigarettes and vaporisers as a replacement for tobacco leaf cigarettes has been significant. As this young industry becomes more regulated, improved manufacturing, development and quality assurance testing will be required, e.g., for quality, health and authenticity considerations.

Various embodiments described herein are directed to methods of and apparatus for mass spectrometry, in which an electronic cigarette (“e-cigarette”) or other similar vaporising or atomising device (e.g., “vaporiser”) is activated (e.g., turned ON) such that the electronic cigarette or other device produces an aerosol, smoke, vapour and/or droplets, and then the aerosol, smoke, vapour and/or droplets is mass analysed. The aerosol, smoke, vapour and/or droplets may be characterised based on the mass analysis.

In various embodiments, the vapour or smoke emitted by an electronic cigarette or other vaporising or atomising device is sampled to give a direct, instantaneous profile of the electronic cigarette vapour or smoke. Furthermore, various embodiments provide a single step sampling technique.

This is in contrast with the typically laborious process of monitoring and measuring the compounds inhaled by cigarette smokers, which requires the “smoking” of cigarettes onto filter paper or into a liquid trap, prior to an extraction step and chromatography-mass spectrometry analysis.

In various embodiments, the aerosol, smoke, vapour and/or droplets emitted from an electronic cigarette or other vaporising or atomising device may be sampled using a Rapid Evaporative Ionisation Mass Spectrometry (“REIMS”) ionisation source, which as described further below, may comprise a modified REIMS ionisation source.

REIMS technology has recently been developed for the real time identification of tissues during surgical interventions. The REIMS method is based on the electrosurgical diathermic evaporation of biological tissues to form a partially charged, high organic content aerosol or surgical smoke. The aerosol that is formed is directly introduced into an atmospheric interface of an atmospheric pressure ionisation mass spectrometer.

The Applicants have found that REIMS technology can sample the aerosol, smoke, vapour and/or droplets emitted from an electronic cigarette or other vaporising or atomising device in the same way that it samples the aerosol from an electrosurgical cutting. Moreover, the REIMS source provides a simple and fast method for analysing the chemical content of the vapour emitted from electronic cigarettes and similar vaporising or atomiser devices.

In various embodiments, the mouthpiece of an electronic cigarette or other similar vaporising or atomising device may be positioned in a sampling or inlet port for a mass spectrometer, which may comprise a REIMS ionisation source. The sampling or inlet port may be designed to accommodate the electronic cigarette or other device, e.g., the mouthpiece of the electronic cigarette or other device. An electronic cigarette liquid or solution (“e-liquid”) may be loaded into the electronic cigarette or other similar vaporising or atomising device, prior to or after positioning the device in the sampling or inlet port.

Alternatively, an electronic cigarette liquid or solution may be provided to or loaded into a “standalone” vaporising or atomising device, which may be coupled to a sampling or inlet port of a mass spectrometer. Thus, various embodiments may be performed using devices or products configured for use by a user to inhale an aerosol, smoke, vapour and/or droplets, as well as devices not configured for use by a user to inhale the emitted aerosol, smoke, vapour and/or droplets (e.g., experimental apparatus).

In various embodiments, the sampling or inlet port may be part of the mass spectrometer (i.e., may be a sampling or inlet port of the mass spectrometer) or may be connected to the mass spectrometer, e.g., by tubing or otherwise. The sampling or inlet port may be connected, directly or via one or more other stages, to a mass analyser of the mass spectrometer.

An airtight seal may be generated between the electronic cigarette or other device and the sampling or inlet port.

Where the electronic cigarette or other device comprises a switch or button for activating the electronic cigarette or other device, the electronic cigarette or other device may be turned ON by activating (e.g., pushing or otherwise) the switch or button. An automated (e.g., computer controlled) actuator may be provided for this purpose.

Additionally or alternatively, where the electronic cigarette or other device is configured so as to be activated by a user sucking air through the electronic cigarette or other device, then the electronic cigarette or other device may be turned ON by causing a gas such as air or nitrogen (e.g., a pulse of gas) to pass through the electronic cigarette or other device. One or more gas pressure initiators may be provided for this purpose.

Thus, modifications to a known REIMS ionisation source according to various embodiments may include the provision of a sampling port to accommodate the mouthpiece of an electronic cigarette or other device, and one or more gas pressure initiators for “firing” variants that require the suction of a user to activate the device (e.g., by initiating a heating mechanism of the device). These modifications allow an electronic cigarette to be initiated as it would be by a user, with the vapour then being taken up into the REIMS source.

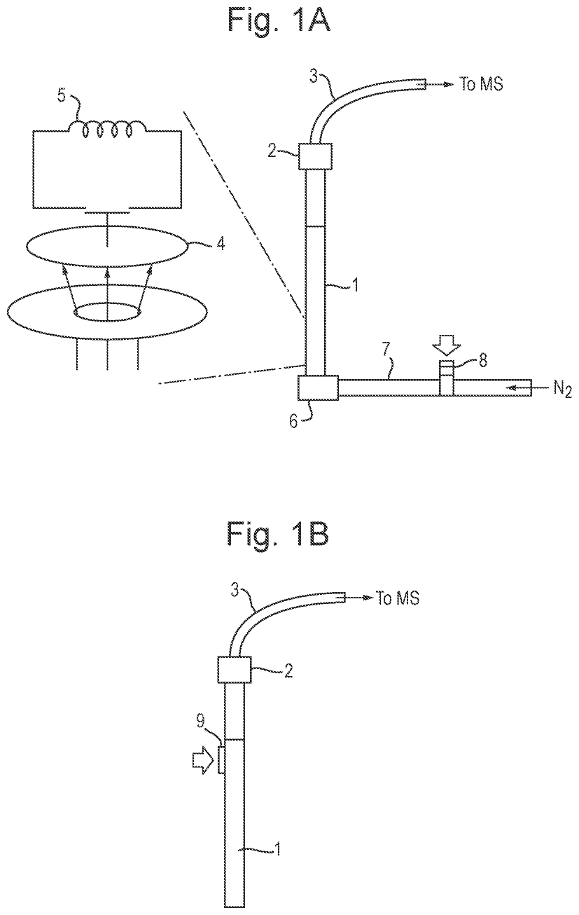

FIGS. 1A and 1B illustrate the activation of two types of electronic cigarette, and their coupling to a mass spectrometer in accordance with various embodiments. As shown in FIG. 1A an airtight gasket 2 may be provided which holds an electronic cigarette 1 in place, e.g., in a manner similar to its intended use. A tube 3 may be provided which connects the electronic cigarette 1 to a mass spectrometer (“MS”) (not shown) via the gasket 2.

FIG. 1A illustrates an electronic cigarette 1 that is configured to be triggered by the suction of a user, e.g., so as to mimic a “normal” cigarette. In this case, the electronic cigarette may comprise an air flow triggered switch 4 which may be configured to close a circuit when sufficient airflow passes through the electronic cigarette 1, e.g., when air is drawn through the device by the suction of a user. The closing of the switch 4 may cause a filament 5 to heat up and vaporise electronic cigarette solution (“e-liquid”) within the device.

In this case, the device may be triggered by passing a sharp blast of gas (i.e. a pressurised gas flow) through the device, i.e. from the opposite end of the device to the mouthpiece, e.g., from a specifically designed addition to a known REIMS source. For example, an airtight gasket 6 may connect the electronic cigarette 1 to a tube 7, wherein the tube 7 may be provided with gas by opening a valve 8. The valve 8 may be opened manually, or an actuator (not shown) may be provided to do this. This arrangement allows such devices to be sampled in an automated and reproducible manner.

FIG. 1B illustrates an electronic cigarette 1 that is configured to be triggered by pressing a button 9 to complete a circuit, e.g., so as to cause a filament to heat up and vaporise electronic cigarette solution within the device. In this case, the device may be initiated by pressing the button 9. This may be done manually, or an actuator (not shown) may be provided to do this.

In the various embodiments described herein, the electronic cigarette or other device may be activated such that electronic cigarette solution (or “e-liquid”) within (or provided to) the electronic cigarette or other device is vaporised so as to produce an aerosol, smoke, vapour and/or droplets.

The resulting aerosol, smoke, vapour and/or droplets may be passed to the mass spectrometer and then mass analysed, e.g., by generating analyte ions and mass analysing the analyte ions or ions derived from the analyte ions (such as fragment ions derived from the analyte ions).

In various embodiments, the aerosol that is formed may be directly introduced into an atmospheric interface of an atmospheric pressure ionisation mass spectrometer. Aspiration of the aerosol, smoke, vapour and/or droplets into the mass spectrometer may be facilitated using a Venturi pump, e.g., driven by nitrogen or air.

In some embodiments, the aerosol, smoke, vapour and/or droplets may be directly mass analysed, e.g., by directly introducing the aerosol, smoke, vapour and/or droplets into a mass analyser. Additionally or alternatively, ionisation of neutrals molecules in the aerosol, smoke, vapour and/or droplets may be used to enhance the ion yield.

In this latter regard, electrospray and corona discharge methods may be used. For example, secondary electrospray ionisation, fused droplet electrospray ionisation and extractive electrospray ionisation may be used to increase the ion yield. These three techniques are similar in the sense that electrically charged solvent droplets are fused with aerosol particles in the gas phase and the resulting fused droplets undergo an electrospray-like ionisation process.

It is also possible to enhance ionisation by facilitating the collision of the aerosol particles with a collision surface in a vacuum region of the mass spectrometer. According to an embodiment the aerosol particles enter the analyser at an atmospheric interface and are accelerated into a vacuum region of the analyser in the free jet regime. The aerosol particles accelerated by the free jet may then directed be onto a collision surface, that may or may not be heated, causing the ion yield to be enhanced.

Thus, in various embodiments, the aerosol, smoke, vapour and/or droplets emitted from an electronic cigarette or other device may be passed to a vacuum chamber of a mass spectrometer (via the sampling or inlet port) whereupon the smoke or aerosol is ionised upon impacting a collision surface which may be heated. The resulting analyte ions may then be mass analysed.

The mass spectrometer may include a modified atmospheric interface which may include a collision surface which may be positioned along and adjacent to the central axis of the large opening of a StepWave® ion guide. As will be understood by those skilled in the art, a StepWave® ion guide comprises two conjoined ion tunnel ion guides. Each ion guide comprises a plurality of ring or other electrodes wherein ions pass through the central aperture provided by the ring or other electrodes. Transient DC voltages or potentials may be applied to the electrodes. The StepWave® ion guide is based on stacked ring ion guide technology and is designed to maximise ion transmission from the source to the mass analyser. The device allows for the active removal of neutral contaminants thereby providing an enhancement to overall signal to noise. The design enables the efficient capture of the diffuse ion cloud entering a first lower stage which is then may focused into an upper ion guide for transfer to the mass analyser.

The collision surface located within a vacuum chamber of the mass spectrometer may facilitate efficient fragmentation of molecular clusters formed in the free jet region of the atmospheric interface due to the adiabatic expansion of gas entering the vacuum chamber and the resulting drop of temperature.

The surface-induced dissociation of supramolecular clusters may improve the signal intensity and also may alleviate the problems associated with the contamination of ion optics.

A matrix comprising an organic solvent such as isopropanol may be added to the aerosol, smoke, vapour and/or droplets, e.g. at the atmospheric pressure interface of the mass spectrometer. The mixture of aerosol, smoke, vapour and/or droplets and organic solvent may then be arranged to impact upon the (optionally heated) collision surface, e.g. as described above. The aerosol, smoke, vapour and/or droplets may be caused to ionise upon impacting the collision surface resulting in the generation of analyte ions. The ionisation efficiency of generating the analyte ions may be improved by the addition of the organic solvent. However, the addition of an organic solvent is not essential.

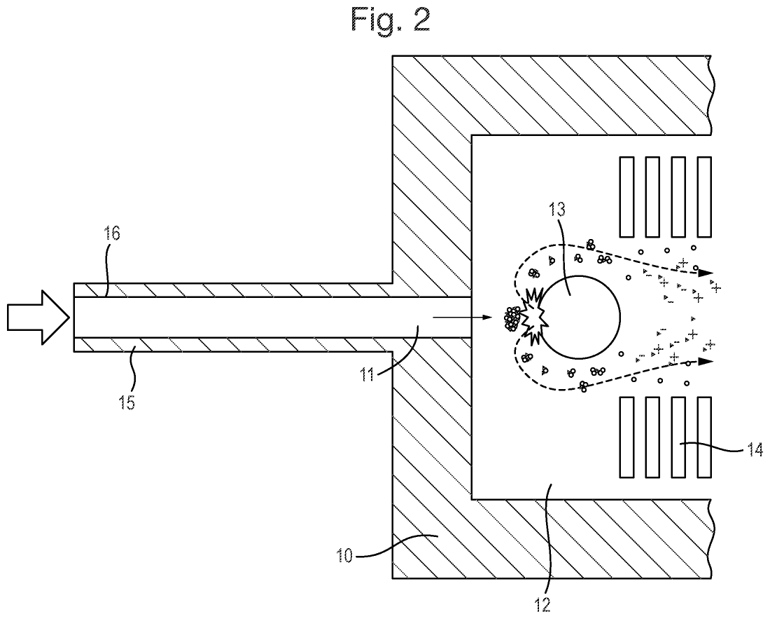

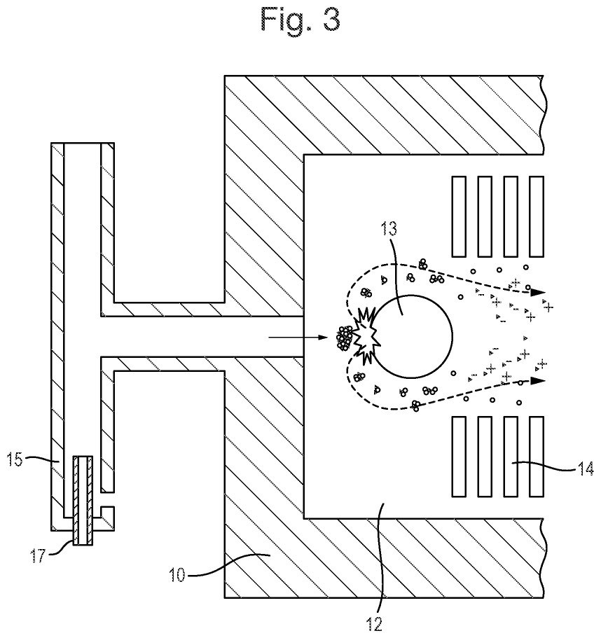

FIG. 2 illustrates schematically an embodiment. The embodiment shown and described with reference to FIG. 2 may comprise an ion analyser or mass spectrometer 10 having an inlet 11, a vacuum region 12, a solid collision surface 13 and ion optics 14 arranged within the vacuum region 12. The arrangement may also comprise a sample transfer tube 15. The sample transfer tube 15 may have an inlet 16 for receiving aerosol sample from a sample being analysed and an outlet that is connected to the inlet 11 of the ion analyser or mass spectrometer 10.

A sample may be generated, e.g., by activating an electronic cigarette 1, as described above. The aerosol particles emitted from the electronic cigarette 1 may then be introduced into the inlet 16 of the sample transfer tube 15. The aerosol particles are drawn towards the inlet 11 of the ion analyser or mass spectrometer 10 by a pressure differential caused by the vacuum chamber 12 being at a lower pressure than the inlet 16 to the tube 15.

The particles may exit the sample transfer tube 15 and pass into the inlet 11 of the ion analyser or mass spectrometer 10. The particles then enter into a decreased pressure region 12 and gain substantial linear velocity due to the adiabatic expansion of gas entering the vacuum region 12 from the sample transfer tube 15 and due to the associated free jet formation. The accelerated particles may impact on a solid collision surface 13, where the impact event fragments the particles, leading to the eventual formation of gas phase ions of the molecular constituents of the aerosol sample. The solid collision surface 13 may be controlled and maintained at a temperature that is substantially higher than the ambient temperature.

The gas phase ions of the molecular constituents of the aerosol sample may be transferred by the ion optics 14 to an analysis region of the ion analyser or mass spectrometer 10. The ions may be guided to the analysis region by applying voltages to the ion optics 14. The ions may then be analysed by the ion analyser or mass spectrometer 10. As a result of the analysis, chemical information about the sample may be obtained.

FIG. 3 illustrates schematically an embodiment that is substantially similar to that shown and described above in relation to FIG. 2, except that the sample is delivered by a fluid/liquid transfer pump or a Venturi pump 17.

In various embodiments, the aerosol, smoke, vapour and/or droplets may be mass analysed in positive ion mode and/or negative ion mode.

The mass analysis may be used to produce one or more mass spectra for the electronic cigarette or other device being analysed. The one or more mass spectra may be used in the characterisation of the aerosol, smoke, vapour and/or droplets or may be the result of the characterisation. Thus, the step of characterising the aerosol, smoke, vapour and/or droplets based on the mass analysis may comprise (a computer) generating one or more mass spectra based on the mass analysis.

Characterising the aerosol, smoke, vapour and/or droplets may comprise characterising the aerosol, smoke, vapour and/or droplets that would be inhaled by a user and/or characterising the electronic cigarette or other device and/or characterising the electronic cigarette solution.

Thus, for example, one or more contaminants in the aerosol, smoke, vapour and/or droplets may be detected and identified, e.g., using the mass spectrum. Additionally or alternatively, the composition of the electronic cigarette solution (“e-liquid”) may be identified or differentiated, e.g., based on the mass spectrum.

In this regard, the Applicants have found that sampling the aerosol, smoke, vapour and/or droplets according to various embodiments produces an information rich and characteristic mass spectrum in both positive and negative ion mode.

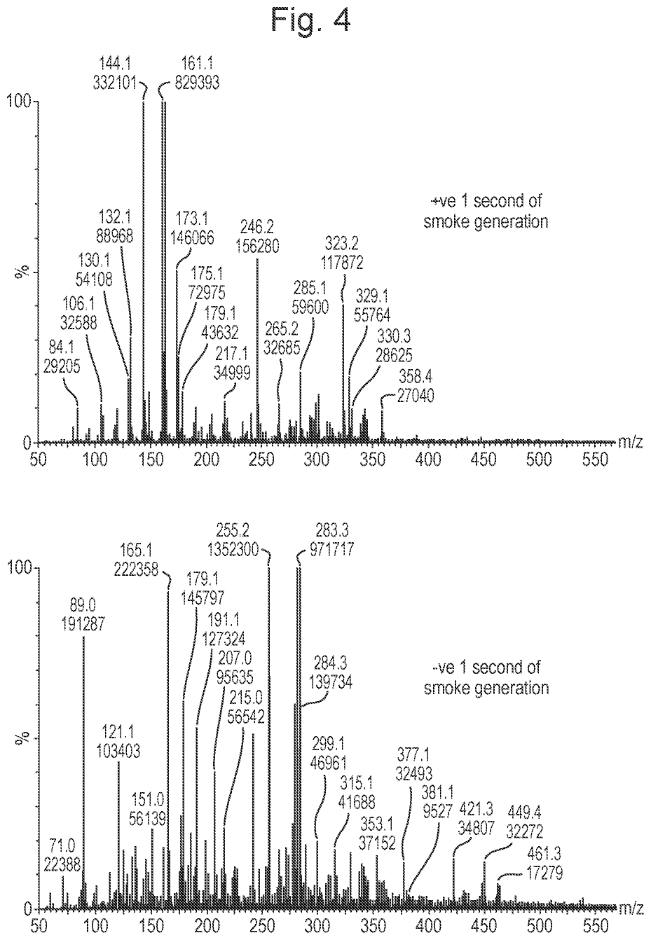

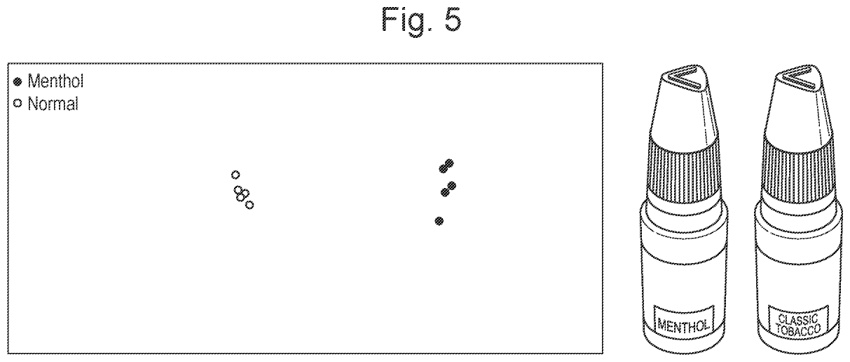

FIG. 4 shows positive and negative ion spectra from a single “burn” of an electronic cigarette. In FIG. 4, the intensity scale is zoomed-in due to dominant mass to charge ratio (“m/z”) peak at m/z 163 in positive ion mode (which corresponds to nicotine) and at m/z 255 and 281 in negative ion mode (which correspond to palmitic and oleic acid, respectively).

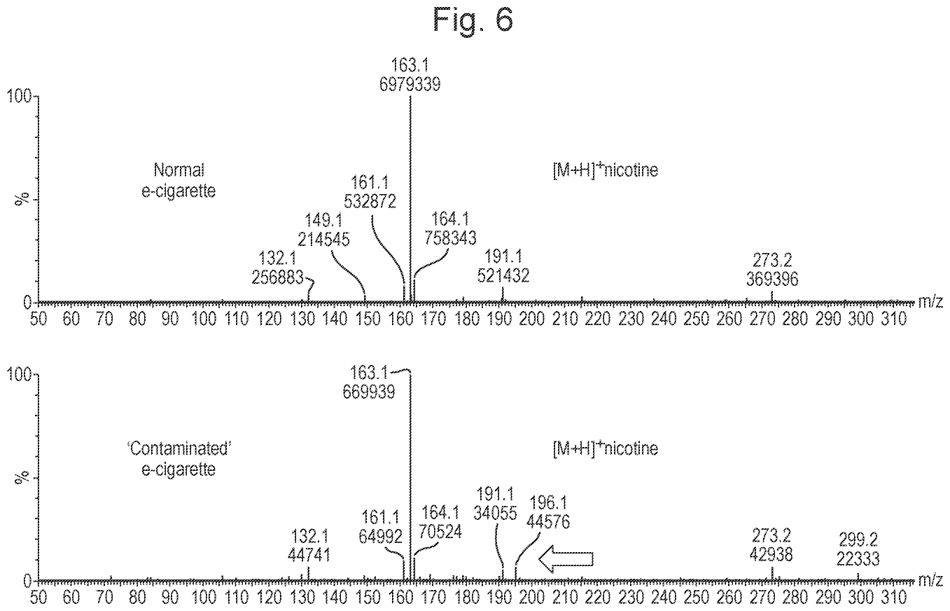

Different blends of solution can be differentiated based on their chemical profiles. This is illustrated in FIG. 5, which shows the results of a Principle Component Analysis (“PCA”) of mass spectra for two different e-cigarette liquids (in this case, “menthol” and “classic tobacco”). In FIG. 5, five sample points for each liquid are shown. The different e-cigarette liquids can be clearly differentiated.

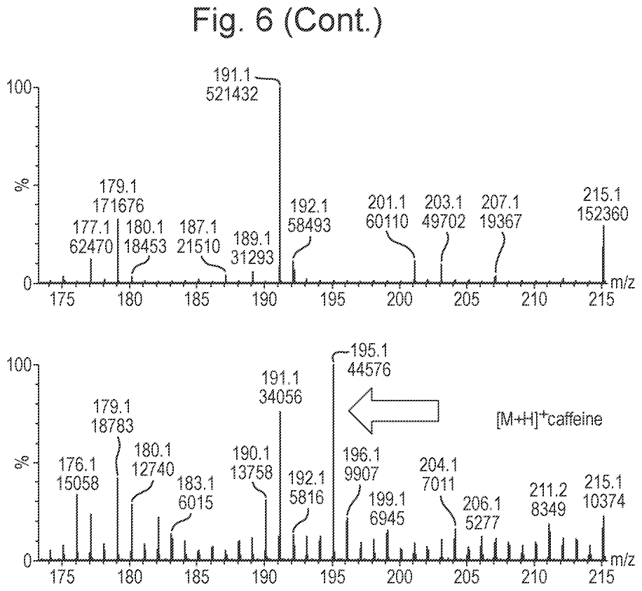

Additionally or alternatively, contaminants can be detected, e.g., as outliers from the normal population of previous samples. This is illustrated by FIG. 6, which shows positive ion mode spectra of a single burn of an e-cigarette. The top spectra in FIG. 6 are from an unadulterated e-liquid, and the lower spectra in FIG. 6 are from the same liquid with a trace amount of caffeine added to mimic a contamination.

The methods described herein may be applied to a single electronic cigarette or other device, but may also be applied to plural electronic cigarettes or other devices, e.g., in series or concurrently.

In this latter case, a plurality of electronic cigarettes (“e-cigarettes”) or other vaporising or atomising devices may be activated such that each of the plurality of electronic cigarettes or other devices concurrently emits aerosol, smoke, vapour and/or droplets. The aerosol, smoke, vapour and/or droplets emitted from each of the plurality of electronic cigarettes or other devices may be mass analysed, and then the plurality of electronic cigarettes or other devices may be characterised based on the mass analysis.

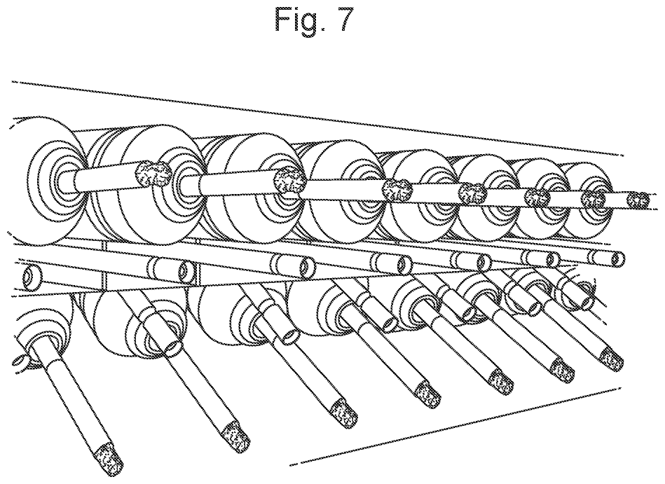

Such an arrangement is particularly useful for “industry scale” characterisation of electronic cigarettes and/or electronic cigarette solutions, e.g., in an analogous manner to conventional cigarette smoke sampling devices, as shown in FIG. 7.

Thus, in various embodiments, the techniques described herein may be used as part of a batch control or monitoring system, and/or a quality control system.

The various embodiments described herein may also be used in conjunction with tasting panels, e.g., to fine tune the composition of new formulations of electronic cigarette solutions.

One or more, any or all of the steps of the various embodiments described herein may be automated (e.g., computer controlled), e.g., without user interaction. Thus, for example, the electronic cigarette(s) or other device(s) may be automatically loaded into a sampling port (e.g. robotically), automatically activated, automatically mass analysed and/or automatically characterised, etc.

According to various embodiments, the spectrometer may comprise a control system. The control system may be configured to control the operation of the spectrometer, e.g. in the manner of the various embodiments described herein. The control system may comprise suitable control circuitry that is configured to cause the spectrometer to operate in the manner of the various embodiments described herein. The control system may also comprise suitable processing circuitry configured to perform any one or more or all of the necessary characterising, processing and/or post-processing operations in respect of the various embodiments described herein.

Although the above embodiments have been described in terms of mass analysing and characterising an aerosol, smoke, vapour and/or droplets produced from an electronic cigarette liquid, the same vaporising principle could be used to analyse a liquid of interest, e.g., that is suspended with a matrix (e.g., propylene glycol). Thus, in various embodiments, the step of activating the vaporising or atomising device may comprise activating the device such that an analyte suspended in a matrix is vaporised by the device so as to produce the aerosol, smoke, vapour and/or droplets.

Furthermore, the techniques of the various embodiments described herein may be used to analyse conventional cigarettes, e.g., by incorporating a REIMS source into a smoking machine, such as the one shown in FIG. 7. This may reduce analysis times, by removing the sample extraction and preparation steps of conventional testing techniques (where, e.g., conventional cigarettes are tested by a multi-step method that requires analytes to be trapped onto filter paper or liquid traps, as described above).

Although the present invention has been described with reference to preferred embodiments, it will be understood by those skilled in the art that various changes in form and detail may be made without departing from the scope of the invention as set forth in the accompanying claims.

1. A method of analysing a sample comprising:
activating an electronic cigarette (“e-cigarette”) or other vaporising or atomising device such that said electronic cigarette or other device emits aerosol, smoke, vapour and/or droplets;
mass analysing said aerosol, smoke, vapour and/or droplets; and
characterising said aerosol, smoke, vapour and/or droplets based on said mass analysis.

2. A method as claimed in claim 1, wherein activating said electronic cigarette or other device comprises activating an activation switch or button of said electronic cigarette or other device.

3. A method as claimed in claim 1, wherein activating said electronic cigarette or other device comprises causing a gas to pass through said electronic cigarette or other device.

4. A method as claimed in claim 3, wherein activating said electronic cigarette or other device comprises causing a pulse of gas to pass through said electronic cigarette or other device.

5. A method as claimed in claim 1, wherein activating said electronic cigarette or other device comprises activating said electronic cigarette or other device such that an electronic cigarette solution (“e-liquid”) is vaporised by said electronic cigarette or other device so as to produce said aerosol, smoke, vapour and/or droplets.

6. A method as claimed in claim 1, further comprising positioning said electronic cigarette or other device in a sampling or inlet port, wherein said sampling or inlet port is arranged and adapted to accommodate said electronic cigarette or other device.

7. A method as claimed in claim 6, further comprising positioning a mouthpiece of said electronic cigarette or other device in said sampling or inlet port, wherein said sampling or inlet port is arranged and adapted to accommodate a mouthpiece of an electronic cigarette or other device.

8. A method as claimed in claim 6, further comprising providing an airtight seal between said electronic cigarette or other device and said sampling or inlet port.

9. A method as claimed in claim 1, wherein mass analysing said aerosol, smoke, vapour and/or droplets comprises generating analyte ions from said aerosol, smoke, vapour and/or droplets.

10. A method as claimed in claim 9, wherein generating analyte ions from said aerosol, smoke, vapour and/or droplets comprises causing said aerosol, smoke, vapour and/or droplets to impact a collision surface.

11. A method as claimed in claim 9, wherein mass analysing said aerosol, smoke, vapour and/or droplets comprises mass analysing said analyte ions or ions derived from said analyte ions.

12. A method as claimed in claim 1, wherein characterising said aerosol, smoke, vapour and/or droplets comprises determining the chemical composition of said aerosol, smoke, vapour and/or droplets based on said mass analysis.

13. A method as claimed in claim 1, wherein characterising said aerosol, smoke, vapour and/or droplets comprises identifying one or more contaminants in said aerosol, smoke, vapour and/or droplets based on said mass analysis.

14. A method as claimed in claim 1, wherein characterising said aerosol, smoke, vapour and/or droplets comprises identifying one or more compositions of electronic cigarette solution (“e-liquid”) based on said mass analysis.

15. A method as claimed in claim 1, further comprising:
activating a plurality of electronic cigarettes (“e-cigarettes”) or other vaporising or atomising devices such that each of said plurality of electronic cigarettes or other devices emits aerosol, smoke, vapour and/or droplets;
mass analysing said aerosol, smoke, vapour and/or droplets emitted from each of said plurality of electronic cigarettes or other devices; and
characterising said plurality of electronic cigarettes or other devices based on said mass analysis.

16. A method as claimed in claim 1, wherein said method is performed automatically without user interaction.

17. Apparatus for analysing a sample comprising:
one or more first devices arranged and adapted to activate an electronic cigarette (“e-cigarette”) or other vaporising or atomising device such that said electronic cigarette or other device emits aerosol, smoke, vapour and/or droplets;
a mass analyser arranged and adapted to mass analyse said aerosol, smoke, vapour and/or droplets; and
one or more second devices arranged and adapted to characterise said aerosol, smoke, vapour and/or droplets based on said mass analysis.

18. (canceled)

19. (canceled)

20. (canceled)

21. (canceled)

22. Apparatus as claimed in claim 17, further comprising a sampling or inlet port, wherein said sampling or inlet port is arranged and adapted to accommodate said electronic cigarette or other device and wherein said sampling or inlet port is arranged and adapted to provide an airtight seal between said electronic cigarette or other device and said sampling or inlet port.

23. (canceled)

24. (canceled)

25. Apparatus as claimed in claim 17, further comprising one or more devices arranged and adapted to generate analyte ions from said aerosol, smoke, vapour and/or droplets, and a collision surface, wherein said apparatus is arranged and adapted to cause said aerosol, smoke, vapour and/or droplets to impact said collision surface to generate analyte ions.

26. (canceled)

27. (canceled)

28. (canceled)

29. (canceled)

30. (canceled)

31. (canceled)

32. (canceled)

33. A method of analysing a sample comprising:
causing a cigarette to emit smoke;
impacting said smoke with a collision surface to produce analyte ions;
mass analysing said smoke, said analyte ions and/or ions derived from said analyte ions; and
characterising said smoke based on said mass analysis.

34. (canceled)
